(19) United States
(12) Patent Application Publication
(10) Pub. No.: US 2006/0035968 A1
(43) Pub. Date: Feb. 16, 2006

(54) Composition for preventing cancer comprising 2'-benzoyl-oxycinnamaldehyde

(75) Inventors: Byoung-Mog Kwon, Daejeon (KR); Dae-Yeul Yu, Daejeon (KR); Eun-Yi Moon, Yongin-si (KR); Kwang-Hee Son, Daejeon (KR); Dong Cho Han, Daejeon (KR)

Correspondence Address: GREENBLUM & BERNSTEIN, P.L.C., 1950 ROLAND CLARKE PLACE, RESTON, VA, 20191, US

(73) Assignee: Korea Research Institute of Bioscience and Biotechnology, Daejeon (KR)

(21) Appl. No.: 11/019,977

(22) Filed: Dec. 23, 2004

(30) Foreign Application Priority Data
Aug. 13, 2004 (KR) 10-2004-0063943

(51) Int. Cl. A61K 31/235

(52) U.S. Cl. 514/532

(57) ABSTRACT

Disclosed is a composition for preventing cancer comprising a compound represented by Chemical Formula 1, 2′-benzoyloxycinnamaldehyde (BCA). BCA has the effects of delaying tumor incidence and increasing immune cells in a transgenic mouse overexpressing H-ras oncogene playing a critical role in tumor cell growth. Thus, BCA is useful as a cancer preventive drug.

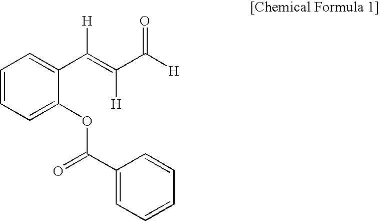

### BACKGROUND OF THE INVENTION

1. Field of the Invention

The present invention relates to a composition for preventing cancer comprising 2′-benzoyloxycinnamaldehyde.

2. Description of the Prior Art

Tumors are produced and maintained by complex interaction between tumors and a host regulating cell proliferation, angiogensis and immunoseparation (J. C. Wasson. et al., Oncogene amplification in pediatric brain tumors, Cancer Res., 1990, 50, 2987-2990). Most of these interactions between the host and tumors are regulated by oncogenes and tumor suppressor genes, which play critical roles in the transformation of normal cells into tumors.

To date, over thirty oncogene families have been identified. These genes are classified according to their subcellular positions and expected mechanisms of their protein products. Ras oncogene belongs to a gene family that encodes associated proteins which is located in plasma membrane. Three ras genes, H-ras, K-ras and N-ras, were identified in the mammalian genome. Ras genes are mostly activated by point mutations, which occur by single base-pair mutations at codons 12, 13 and 61. The most common mutation of activated Ras in human tumors occurs at codon 12 of H-ras gene. A single base mutation from GGC to GTC occurring in this position results in the substitution of glycine in the GTPase regulatory domain of Ras with valine. This single amino acid substitution leads to a loss of GTPase activity of H-ras, and thus, H-ras protein remains in an active state because it cannot convert GTP to GDP. This long-lasting activation of H-ras affects nuclear genes through signal transduction pathways (raf, MAPK kinase, ERK, etc.) and eventually stimulates cellular differentiation and proliferation.

Ras mutations are found in about 30% of all types of human cancer, and the incidence varies according to the type of tumors. The highest incidence of about 75% to 100% is found in pancreatic cancer, and other incidences include about 50% for colon cancer, about 30% for lung cancer and about 50% for thyroid cancer (Bos. J. L., Ras oncogenes in human cancer: a review, Cancer Res. 49, 4682-4689, 1989).

Recent studies teach that H-ras gene is related with both production and maintenance of solid tumors (L. Chin. et al., Essential role for oncogenic Ras in tumour maintenance, Nature, 400, 468-472, 1999). H-ras is an oncogene that is present in high frequency in an active form in the tissue of liver cancer etc. Liver caner is the most common cancer in the world, and is typically classified into two major categories: cancer arisen from the liver itself, generally called “primary liver cancer”; and liver cancer spread to the liver from another organ (metastatic cancer), which is responsible for most malignant liver tumors. Liver cancer accounts for 11.6% of registered cancer patients and 17.3% of cancer deaths. Unlike in America or the West, liver cancer occurs in high frequency in Asia and Africa and causes significant health problems. In particular, liver cancer ranks second in male cancer incidence, and its incidence rate is four times higher in males than in females. Hepatocirrhosis often precedes liver cancer. Liver cancer usually occurs between the ages of 40 to 50 in the West, and between the ages of 30 to 40 in Asia and Africa. In addition, mutational activation of H-ras was observed in about 30% of nodular melanin-defective melanomas, and H-ras is known to stimulate and regulate expression of VEGF essential for tumor vascularization (N. Bardeesy. et al., Dual inactivation of RB and p53 pathways in RAS-induced melanomas. Mol. Cell. Biol., 21, 2144-2153, 2001).

Efforts were made to develop transgenic mice as a tumor model using H-ras gene (Tremblay et al., Mol. Cell. Biol., 9, 854-859, 1989; Gilbert et al., Int. J. Cancer, 73, 749-756, 1997). However, these transgenic mice were not suitable as an animal tumor model for in vivo validation of efficacy of drug candidates for cancer therapy because they died at early stages, were sterile or had a low potential to induce liver cancer. In this regard, the present inventors established a transgenic mouse overexpressing H-ras using H-ras gene, which develops liver cancer at a proper time, does not cause lesions in other organs and propagates well, and filed a patent application for a patent for the transgenic mice (Korean Pat. Application No. 10-2002-0067876).

On the other hand, BCA (2′-benzoyloxycinnamaldehyde), which is a member of cinnamaldehydes, was reported to inhibit farnesyl-protein transferase (Cinnamaldehyde inhibits lymphocyte proliferation and modulates T-cell differentiation, Int J Immunopharmacol, 20(11), 643-60, 1998 Nov), and is also known as a drug candidate for cancer therapy in propagated tumor cells (Dong Cho Han et al., J. Biological Chemistry, 279(8), 6911-6920, 2004). However, to date, there has been no report mentioning BCA as a potential preventive agent against cancer.

With this background, the present inventors found that, when BCA was administered to the above-mentioned H-ras-overexpressing mouse model to induce cancer, BCA delayed carcinogenesis and increased the immune activity of the mouse. Also, based on the previous animal test result indicating that BCA has no in vivo toxicity, the present inventors found that BCA has the potential to effectively prevent cancer without toxicity, leading to the present invention.

### SUMMARY OF THE INVENTION

It is therefore an object of the present invention to provide a composition for preventing cancer comprising a compound represented by Chemical Formula 1.

It is another object of the present invention to provide a method of preventing carcinogenesis, which is based on administering a compound represented by Chemical Formula 1.

### BRIEF DESCRIPTION OF THE DRAWINGS

The above and other objects, features and other advantages of the present invention will be more clearly understood from the following detailed description taken in conjunction with the accompanying drawings, in which:

FIGS. 1A and 1B are photographs showing liver cancer incidence (1A) and vascular invasion of tumor cells (1B) in a six-month-old, H-ras 12V-overexpressing mouse;

FIG. 2A is a photograph showing that administration of 2′-benzoyloxycinnamaldehyde (BCA) inhibits tumor cell growth in the liver of a H-ras 12V-overexpressing mouse, leading to the inhibition of tumor progression to malignant tumors, and FIG. 2B is a photograph showing lymphocytes recruited into the liver tissue, which are indicated by an arrow; and

FIGS. 3A and 3B are graphs showing the increased splenocyte cell number by BCA in a H-ras 12v transgenic mouse (Tg) (3A) and the increased response to a T lymphocyte proliferation inducer Con A (concanavalin A) (3B).

### DETAILED DESCRIPTION OF THE INVENTION

In an aspect, the present invention relates to a composition for preventing cancer comprising a compound represented by Chemical Formula 1, below.

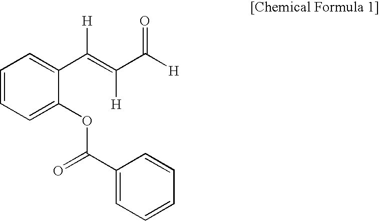

BCA (2′-benzoyloxycinnamaldehyde) represented by Chemical Formula 1 may be prepared from a natural source, for example, the stem bark of Cinnamomum cassia, by a known extraction method, or synthesized by a method widely known in the art.

When BCA is to be isolated from the stem bark of C. cassia, for example, as described in Korean Pat. Application No. 1999-32142, BCA of Chemical Formula 1 is prepared by a method including extracting the stem bark of C. cassia with a mixture of chloroform and acetone, concentrating the resulting extract and extracting the concentrate with a sodium hydroxide solution, acidifying a basic layer and extracting an acidified product with methylene chloride, concentrating the methylene chloride extract, and subjecting the concentrate to silica gel chromatography and high speed liquid chromatography to purify 2′-hydroxycinnamaldehyde (HCA); and, subsequently, another method including reacting the purified HCA with triethylamine and benzoyl chloride in CH₂Cl₂.

BCA is a derivative of HCA isolated from an edible plant, Cinnamomum cassia, which has been used in Chinese medicine. BCA is safe with no risk of side effects and toxicity, and this safety was demonstrated by a recent animal test showing that BCA has no toxicity in vivo (J. Toxicol. Pub. Health, 2003, 19, 259-266).

The present composition comprising BCA has the cancer preventive effect of delaying carcinogenesis and increasing the number of immune cells. The cancer preventive effect of BCA is distinguishable from the cancer therapeutic effect of BCA, which was already demonstrated before the present invention. Dong Cho Han et al. in J. Biological Chemistry, 279(8), 6911-6920, 2004 reported that HCA and BCA has inhibitory effects against farnesyl-protein transferase in vitro, angiogenesis and tumor cell growth, and that reactive oxygen species are major regulators for this BCA activity. In addition, as described in Jae Jun Lee et al., IOVS, 43(9), 3117-3124, 2002 Sep., BCA induces apoptosis in human retinal pigment epithelial (hRPE) cells and has an antiproliferative effect in a proliferative vitreoretinopathy (PVR) model in rabbits.

Typically, the term “anticancer” indicates both therapy and prevention. This term is mostly related to the action as a therapeutic agent of treating developed cancer, and the conventional anticancer effects of BCA indicate only its effect as a cancer therapeutic agent. The commonly used term “cancer inhibitor” refers to the aforementioned cancer therapeutic agent. The cancer preventive effects of substances having anticancer effects should be considered as a different problem from cancer therapy, and these substances may be potential candidates for cancer therapy when their inhibitory effects against carcinogenesis are confirmed using suitable animal models. In other words, distinction should be made between a preventive agent that prevents an existing factor to develop cancer from progressing to cancer and a therapeutic agent that reduces the size of grown cancer or delays its growth.

Before the present invention, BCA was known to have anticancer effects that are related only to therapeutic effects of reducing the size of grown cancer or delaying its growth, and was not known to have a preventive effect against cancer expected to occur.

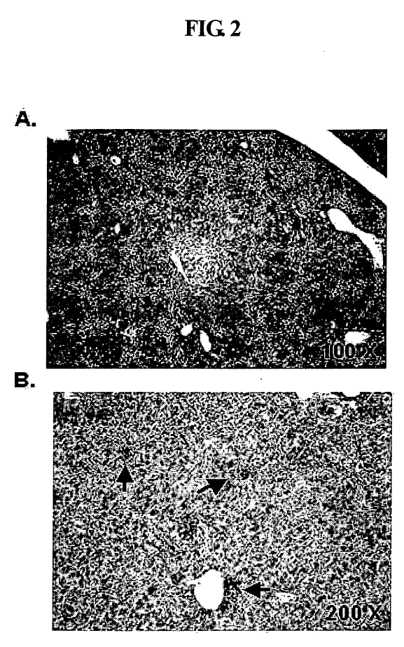

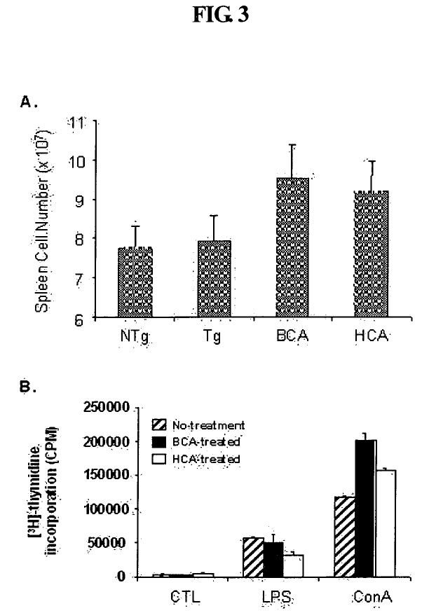

The cancer preventive effect of the BCA compound of the present invention was demonstrated by employing an H-ras oncogene transgenic mouse (accession number: KCTC 10318 BP) developed by the present inventors. The H-ras-transgenic mouse is an animal model of carcinogenesis, which is manipulated to spontaneously develop liver cancer six months after birth. In detail, the H-ras-transgenic mouse is prepared by constructing an expression vector using H-ras cDNA (containing a substitution of glycine at codon 12 with valine) as a reporter gene, that is, constructing an expression vector by inserting into a pBluescript vector a fusion gene including albumin enhancer/promoter, H-ras cDNA (containing a substitution of glycine at codon 12 with valine) and SV40 polyA; purifying the fusion gene except for the pBluescript vector region from the expression vector; microinjecting the purified fusion gene into mouse fertilized eggs; implanting the fertilized eggs into the oviduct of a surrogate mother; and allowing the development of the eggs to provide a carcinogenesis model stably expressing H-ras at low levels where liver cancer is induced after six month of birth without death at early stages by the resulting overexpression of activated H-ras. The present inventors administered BCA to the transgenic mouse before the incidence of liver cancer and investigated whether liver cancer was induced or not in the transgenic mouse. As a result, cancer was induced in a group not administered with BCA, whereas, in another group administered with BCA, carcinogenesis was delayed and the number of immune cells increased (FIGS. 2 and 3). In addition, the BCA administration group displayed the highest T cell response (FIG. 3B).

The Ras proteins act as a molecular switch that is regulated by GTP of 21 kDa and controls many cellular behaviors including proliferation, differentiation, mobility and death. Ras is called “p21” consisting of 189 amino acids. The first 86 amino acids are almost identical between the three Ras genes, and, in the next 79 amino acids, there is a high homology between the three ras genes. The Ras proteins have no sequence similarity only in their C-terminal 24 amino acids. In many tumors, oncogenic mutations occur at positions 12, 13 and 61 of the Ras proteins. These mutations lead to a loss of GTPase activity of Ras, and thus, Ras remains in a GTP-bound state, an active state. This activation of Ras affects nuclear genes through signal transduction pathways and eventually stimulates cellular differentiation and proliferation.

Therefore, the present composition comprising BCA may be used for preventing all types of cancer induced by abnormal overexpression of the Ras proteins. In a preferred aspect, the present composition may be used for preventing cancer induced by H-ras overexpression, including liver cancer, brain tumors, lung cancer, breast cancer, cervical cancer, stomach cancer and colon cancer, but the present invention is not limited to these types of cancer. In particular, the present composition may be preferably used for preventing liver cancer and colon cancer (L. Chin. et al., Essential role for oncogenic Ras in tumour maintenance, Nature, 400, 468-472, 1999).

In another aspect, the present invention relates to a method of preventing cancer, which is based on administering the BCA compound of Chemical Formula 1 to a patient expected to develop cancer.

The term “patient”, as used herein, refers to a subject for administration of the composition of the present invention, that is, an animal expected to develop cancer, preferably a mammalian animal, particularly a human.

In a preferred aspect, the present invention relates to a method of preventing cancer induced by overexpression of Ras proteins, in particular, H-ras overexpression, for example, liver cancer, brain tumors, lung cancer, breast cancer, cervical cancer, stomach cancer and colon cancer. In a more preferred aspect, the present invention relates to a method of preventing liver cancer or colon cancer.

The present composition having cancer preventive effects as described above may comprise, in addition to BCA, pharmaceutically or physiologically acceptable, suitable carriers, excipients, diluents, and the like.

The present composition may be formulated into oral preparations and sterile injectable solutions, such as powders, granules, tablets, capsules, suspensions, emulsions, syrups and aerosols, by commonly used methods according to the intended use, and these preparations may include carriers, excipients and diluents, which are exemplified by lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinylpyrrolidone, water, methylhydroxybenzoate, propylhydroxybenzoate, talc, magnesium stearate and mineral oils.

Solid preparations for oral administration include tablets, pills, powders, granules, capsules, etc. These solid preparations are formulated by mixing the present composition with at least one excipient, for example, starch, calcium carbonate, sucrose, lactose and gelatin. Also, in addition to simple excipients, lubricants such as magnesium stearate or talc are also used. Liquid preparations for oral administration include suspensions, solutions, emulsions, syrups, etc., and may include commonly used, simple diluents such as water and liquid paraffin, and, if desired, may further include various excipients, for example, humectants, sweeteners, aromatics and preservatives.

Preparations for parental administration include sterile aqueous solutions, non-aqueous solutions, suspensions, emulsions, freeze-dried preparations, suppositories, etc. For non-aqueous solutions and suspensions, propylene glycol, polyethylene glycol, vegetable oils such as olive oil, and injectable esters such as ethyloleate may be used. Bases of injectable preparations may include conventional additives, such as solubilizing agents, tonicity adjusting agents, suspending agents, emulsifying agents, stabilizing agents and antiseptics.

Dosage of the composition of the present invention may vary according to the patient's age, sex, body weight, etc., but, typically, the present composition may be administered in a dose of 5-100 mg, preferably, 10-50 mg, and more preferably, 15-30 mg per kg body weight 1 to 3 times per day or every two days. In addition, the dosage of the present composition may increase or decrease according to the administration routes and the patient's pathogenic state, sex, weight, age, etc. Therefore, the above dosage does not limit the scope of the present invention.

The composition of the present invention may be used as a medicine for preventing cancer, as well as be added to functional health foods for maintaining a healthy life. Examples of health foods to which the present composition can be added include various food products, beverages, gums, multivitamin preparations, health functional foods, etc.

Typically, the present composition may be added to a food product or beverage in an amount of 0.01-90 wt %, and preferably 0.1-50 wt %, based on the total weight of the food product or beverage. In particular, the present composition may be added to a health functional beverage in an amount of 0.01-20 g and preferably 0.1-10 g per 100 ml.

A health functional beverage containing the composition of the present invention may further include various sweeteners or natural carbohydrates, which are commonly added to beverages. Examples of the natural carbohydrates include monosaccharides (e.g., glucose, fructose, etc.), disaccharides (e.g., maltose, sucrose, etc.), polysaccharides (e.g., common sugars such as dextrin, cyclodextrin, etc.), and sugar alcohols (e.g., xylitol, sorbitol, erythritol, etc.). Examples of the sweeteners include natural sweeteners (Stevia extracts such as rebaudioside A, glycyrrhizin, thaumatin, etc.) and synthetic sweeteners (saccharin, aspartame, etc.). In addition to the above ingredients, the health functional beverage may further include various nutrients, vitamins, minerals (electrolytes), synthetic and natural perfumes, flavors, colorants, fillers, pectic acid and salts thereof, alginic acid and salts thereof, organic acids, protective colloidal thickeners, pH regulators, stabilizing agents, antiseptics, glycerins, alcohols, carbonating agents used in carbonated beverages. In addition, the present composition may also include natural fruit juice or fruit flesh for preparation of fruit juice drink or vegetable drink. These ingredients may be used singly or in combination.

A better understanding of the present invention may be obtained through the following example which is set forth to illustrate, but is not to be construed as the limit of the present invention.

### EXAMPLE 1

### Effect of BCA on Incidence of Liver Cancer in H-ras-Overexpressing mouse

A neonatal 4-month-old H-ras 12V-overexpressing mouse (KCTC 10318 BP) was intraperitoneally administered with 50 mg/kg of BCA (prepared by reacting 2′-hydroxy-cinnamaldehyde (HCA) isolated from the stem bark of C. cassia with benzoyl chloride in the presence of triethylamine) for ten weeks once every two days. Six months after birth, the liver tissue was excised from the mouse in an aseptic state, fixed with a formaldehyde solution, and embedded in paraffin. The resulting paraffin block was sectioned into a thickness of 4 μm using a microtome (LEICA Company), and tissue sections were attached onto slides. The tissue was then stained with hematoxylin/eosin and observed under a reverse phased microscope (Model: Bx 50, Olympus Company).

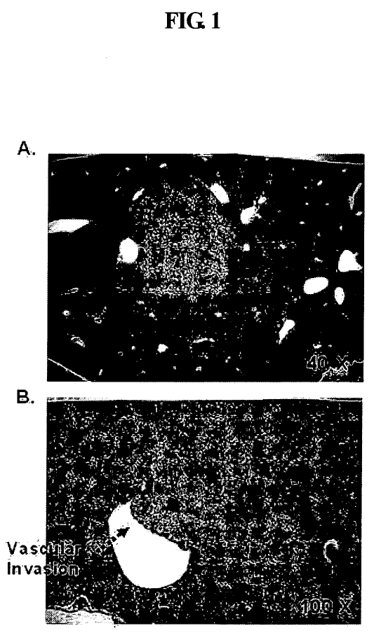

FIGS. 1A and 1B show cancer developed in the liver of the ras-overexpressing mouse. In detail, FIG. 1A shows a typical cancer tissue, and FIG. 1B shows vascular invasion by progressed cancer, which is indicated by an arrow. FIG. 2A shows that, when the H-ras-overexpressing mouse was intraperitoneally injected with BCA, the portion that were progressed to cancer in the liver was remarkably reduced in comparison with FIG. 1A as a control group. FIG. 2B shows lymphocytes recruited into the liver tissue, which are indicated by an arrow.

As apparent from the above results, in the mouse not administered with BCA, liver cancer was spontaneously induced six months after birth (FIGS. 1A and 1B). In contrast, in the mouse administered with BCA, tumor progression to liver cancer was inhibited, and the number of immune cells in the liver was increased (FIGS. 2A and 2B).

### EXAMPLE 2

### Effect of BCA on Proliferation of Splenocytes and T Cell Response in H-ras-Transgenic Mouse

A neonatal 4-month-old H-ras 12V-overexpressing mouse (KCTC 10318 BP) was intraperitoneally administered with 50 mg/kg of BCA and HCA (prepared by extracting the stem bark of C. cassia with organic solvents such as methanol and performing an isolation and purification process by various column chromatography methods) for ten weeks once every two days. Six months after birth, the spleen was excised from the mouse, and a single cell suspension was prepared. Red blood cells were removed from the single cell suspension using a RBC lysis solution (Sigma), and the number of splenocytes was counted under a microscope. FIG. 3A shows increased cell number in the spleen of the H-ras-transgenic mouse (Tg) intraperitoneally injected with BCA and HCA for 10 weeks in comparison with a control group (NTg), wherein cell counting was carried out by harvesting cells from the lymphoid tissue, spleen, and preparing a single cell suspension. The group administered with BSA displayed the largest increase in splenocyte number.

On the other hand, 2×10⁵ splenocytes were aliquotted into a 96-well plate, stimulated with a cell proliferation inducer, LPS (lipopolysaccharide) or ConA (concanavalin A), and cultured for 48 hours. 1 μCi of [³H]-thymidine was added to each well, followed by incubation for 6 hours. Cells were harvested on a nitrocellulose membrane, and radiation activity was measured using a beta scintillation counter (Wallac, Turku, Finland) to investigate proliferation ability of splenocytes. FIG. 3B shows increased cell proliferation by LPS or ConA, which was measured using a radioactive isotope, [³H]-thymidine, in comparison with a control group (CTL). The highest T cell response was shown in the group treated with BCA when stimulating splenocytes with T lymphocyte proliferation inducer, ConA.

As described above, BCA inhibited carcinogenesis and increased the number of immune cells and T cell response in animals expected to develop cancer. These results indicate that BCA is effective in preventing cancer.

As described hereinbefore, BCA has the effects of delaying tumor incidence and increasing immune cells in a transgenic mouse overexpressing H-ras oncogene playing a critical role in tumor cell growth. Also, since BCA is derived from an edible source, such as a plant C. cassia, it is believed to have fewer problems with regard to long-term administration. Thus, BCA is useful as a cancer preventive drug.

1. A composition for preventing cancer in mammals in need thereof comprising a compound represented by Formula 1, wherein the cancer is induced by H-ras overexpression.

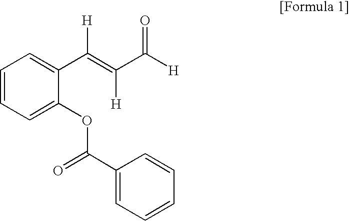

2. (canceled)

3. The composition as set forth in claim 1, wherein the cancer is liver cancer, at least one brain tumor, lung cancer, breast cancer, cervical cancer, stomach cancer or colon cancer.

4. A method of preventing cancer in mammals in need thereof comprising administering an effective amount of a compound represented by Formula 1 before incidence of cancer, wherein the cancer is induced by H-ras overexpression.

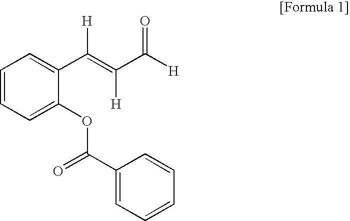

5. (canceled)

6. The method as set forth in claim 4, wherein the cancer is liver cancer, at least one brain tumor, lung cancer, breast cancer, cervical cancer, stomach cancer or colon cancer.

7. A method for preventing the development of cancer in mammals in need thereof comprising administering an effective amount of a compound represented by Formula 1 before incidence of cancer, 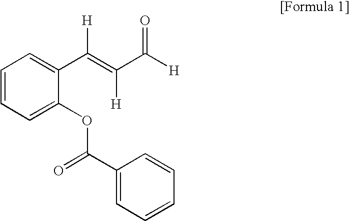
wherein the cancer is induced by H-ras overexpression.

8. The method as set forth in claim 7, wherein the cancer is liver cancer, at least one brain tumor, lung cancer, breast cancer, cervical cancer, stomach cancer or colon cancer.

9. The method as set forth in claim 4, wherein the effective amount of compound represented by Chemical Formula 1 ranges from 5 to 300 mg per kg body weight.

10. The method as set forth in claim 7, wherein the effective amount of compound represented by Chemical Formula 1 ranges from 5 to 300 mg per kg body weight.

11. The method as set forth in claim 7, wherein the cancer is liver cancer.

12. The method as set forth in claim 7, wherein the cancer is at least one brain tumor.

13. The method as set forth in claim 7, wherein the cancer is lung cancer.

14. The method as set forth in claim 7, wherein the cancer is breast cancer.

15. The method as set forth in claim 7, wherein the cancer is cervical cancer.

16. The method as set forth in claim 7, wherein the cancer is stomach cancer.

17. The method as set forth in claim 7, wherein the cancer is colon cancer.
